(19) United States
(12) Patent Application Publication
(10) Pub. No.: US 2022/0105289 A1
(43) Pub. Date: Apr. 7, 2022

(54) METHOD AND SYSTEM FOR AUTOMATING OXYGEN MONITORING AND DOSING IN REAL TIME FOR PATIENT ON OXYGEN THERAPY

(71) Applicant: VANDERBILT UNIVERSITY, Nashville, TN (US)

(72) Inventor: Lisa Hood Lancaster, Nashville, TN (US)

(21) Appl. No.: 17/492,822

(22) Filed: Oct. 4, 2021

Related U.S. Application Data
(60) Provisional application No. 63/086,957, filed on Oct. 2, 2020.

(51) Int. Cl. A61M 16/00; A61M 16/10; G16H 40/63

(52) U.S. Cl. CPC A61M 16/024; A61M 16/1005; G16H 40/63; A61M 2205/3334; A61M 2205/3592; A61M 2230/50; A61M 2205/18; A61M 2205/3553; A61M 2205/502; A61M 2230/63; A61M 2230/20

(57) ABSTRACT

This system and method for automating oxygen monitoring and dosing in real time for a patient on oxygen therapy is disclosed. The system includes a flow regulator coupled to an oxygen source for operably controlling a flow rate of oxygen from the oxygen source to the patient; a wearable device configured to be worn by the patient for measuring physiological parameters of the patient to monitor a physiological status of the patient; and a controller in wireless communication with the wearable device and the flow regulator for receiving the measured physiological parameters from the wearable device, and controlling operations of the flow regulator to regulate the flow rate of oxygen from the oxygen source for providing a desired amount of oxygen to the patient based on the physiological status of the patient.

### CROSS-REFERENCE TO RELATED PATENT APPLICATION

This application claims priority to and the benefit of U.S. Provisional Patent Application Ser. No. 63/086,957, filed Oct. 2, 2020, which is incorporated herein in its entirety by reference.

### FIELD OF THE INVENTION

The invention relates generally to healthcare, and more particularly, to method and system for automating oxygen monitoring and dosing in real time for a patient on oxygen therapy.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

It is estimated that up to 1.5 million people in the United States use supplemental oxygen, but confident numbers are lacking. As more patients are living longer and being diagnosed with lung diseases, the numbers are anticipated to continue to increase. Common causes of hypoxic lung disease include chronic obstructive pulmonary disease (COPD), emphysema, interstitial and fibrotic lung diseases, cardiac disease such as congestive heart failure and ischemic heart disease as well as infectious or inflammatory lung diseases. Oxygen (02) therapy has become standard of care for support of hypoxic lung disease patients and an integral component of care for patients with idiopathic pulmonary fibrosis. Oxygen therapy has been shown in certain pulmonary diseases to improve endurance, walking distance, shortness of breath, improve sleep, improve cognitive function, improve survival, and prevent pulmonary hypertension with subsequent right heart failure.

Despite the large numbers of patients needing both home and portable oxygen, little progress has been made in the development of oxygen devices in the last few decades. Patients remain burdened with cumbersome, heavy tanks, long tubing, frequent need to change tanks if high oxygen flow rates are needed, and the inability to easily change the flow rate on stationary tanks if moving around the home. This can worsen with progressive disease requiring higher flow rates and more oxygen.

Presently, home oxygen prescriptions are written based on the provider's assessment of oxygen needs and prescribed a certain fixed liter flow rate starting at 2 liters per minute. Providers will estimate the needs based on resting oxygen saturations and oxygen saturations during walks in clinic or the hospital while wearing a pulse oximeter. Oxygen is then adjusted or titrated to keep the pulse oximeter saturations in the 90s while walking and at rest. The flow rate of oxygen needed to keep O₂ saturations in the 90s at rest and with walking is then prescribed at a fixed rate for the patient to self-adjust between resting and exertion or sleep.

This becomes logistically problematic in the home due to the reality of patients needing more oxygen when they are more active than when they are at rest. The oxygen concentrator for patients is set up in one room in the house which is usually the bedroom. A patient will walk from the bedroom to the living room or another room in the house and will need a higher flow rate when walking or exerting themselves than when they are at rest. Now they are in the living room planning to sit in a chair so oxygen by prescription should be turned down at rest but they are in the living room far from the oxygen concentrator and regulator that adjusts the flow rate of the oxygen. This continues throughout the day as they move from sitting to room to room. The fixed prescribed rates of oxygen flow for rest and walking do not capture the needs when the patient may be exerting themselves more with other daily activities.

Most patients either over utilize oxygen and place it on the flow rate needed with exertion or under dose the oxygen leaving it on the flow rate needed at rest or somewhere in between. All may be problematic. Higher dosing could develop pro inflammatory free radicals aggravating underlying lung disease. Under dosing oxygen puts patients at risk of pulmonary hypertension and right heart failure, lower endurance, risk of arrythmia, risk of syncope and risk of ischemia. Having the ability to adjust flow rate in real time with the rise and fall of oxygen saturations during rest and with activity would be even more advantageous with portable oxygen. Being able to adjust down the oxygen with sitting as oxygen saturations allow, would enable the patient to conserve the oxygen in the tank resulting in fewer tanks being needed to travel outside the home.

In addition, patients on oxygen in hospitals are adjusted by the nurse who may notice alarms for low oxygen levels or tachycardia and respond by leaving one patient's room and moving to the one with alarms sounding. Due to location and the duties the nurse is performing with another patient, a time delay in adjustment of oxygen occurs. A real time device that sounds the alarm while adjusting the oxygen per software protocol to the flow rate needed by the patient to maintain normoxia would correct hypoxia sooner while avoiding lost time and the numerous complications of hypoxia most notably arrythmia with cardiopulmonary arrest in the acutely ill patient.

Therefore, a heretofore unaddressed need exists in the art to address the aforementioned deficiencies and inadequacies.

### SUMMARY OF THE INVENTION

One of the objectives of this invention is to provide systems and methods for oxygen monitoring with flow rate adjustment of oxygen in real time. One exemplary embodiment of the system includes a bracelet like device that captures pulse oximetry oxygen saturation data in real time and is linked to both an app complete with software providing an oxygen titration protocol that is then linked with the flow rate regulator. This system is wirelessly coupled with home concentrators and portable oxygen tanks to seamlessly provide information and instruction to the regulator to adjust the flow rate to the oxygen needs of the patient. This is the first device of its kind to treat hypoxia in real time and allow for conservation of oxygen without risk of under dosing the patient.

In one embodiment, this is a system for automating oxygen monitoring and dosing in real time for a patient on home or portable oxygen therapy. The system includes a flow regulator coupled to an oxygen source for operably controlling a flow rate of oxygen from the oxygen source to the patient; a wearable device configured to be worn by the patient for measuring physiological parameters of oxygen saturation among others; and a controller application in wireless communication with the wearable device and the flow regulator. After receiving the oxygen saturation data, the controlling operations of the application on the smart phone regulates the flow rate of oxygen from the oxygen source for providing a desired amount of oxygen to the patient based on the physiological status of the patient.

In one embodiment, the physiological parameters comprise at least one of an oxygen saturation (SpO₂), a heart rate, activity level, a respiratory rate, a temperature, and a blood pressure.

In one embodiment, when the SpO₂ is in a predetermined range, the controller generates a signal corresponding to the predetermined range and transmits the signal to the flow regulator to cause the flow regulator to adjust the flow rate of oxygen from the oxygen source corresponding to the predetermined range.

In one embodiment, the SpO₂ is measured by a bracelet pulse oximeter. To ensure accuracy of the pulse oximeter, a regular pulse and repeated SpO₂ is reassessed for 10-20 seconds, prior to the following institution of the protocol.

when the SpO₂ is in a range of 90-95%, the flow rate remains unchanged, and the SpO₂ is rechecked in a time-period of 1-2 minutes;

when the SpO₂ is in a range of 85-89%, the flow rate is adjusted to increase by 1 L/min, and the SpO₂ is rechecked in a time period of 1-2 minutes;

when the SpO₂ is in a range of 81-85%, the flow rate is adjusted to increase by 2 L/min, and the SpO₂ is rechecked in a time period of 1-2 minutes;

when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1-2 min;

when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1-2 min; and

when the SpO₂ is greater than 96%, the flow rate is adjusted to reduce by 1 L/min, and the SpO₂ is rechecked in a time period of 2 minutes, which continues until the SpO₂ is maintained in a range of 90-95%.

In one embodiment, the controller is configured to initiate a call for help, when the patient is in a predetermined physiological status in which the SpO₂ is less than 75% for a predetermined time period, and/or shortness of breath or chest pain is present.

In one embodiment, the controller is configured to store the measured physiological parameters including the SpO₂ and the flow rate over days and weeks in local, or transmit them to a data center, which are accessible by the patient, a professional, and/or a provider.

In one embodiment, the system is configured if more than three alarms occur over a course of a day, the patient is messaged to contact their provider with their oxygen desaturations.

In one embodiment, the controller is configured to average the measured physiological parameters of the patient over a time period of 10-20 sec.

In one embodiment, the controller is a smartphone or tablet.

In one embodiment, the system further comprises a customized application with a graphic user interface (GUI) installed in the smartphone or tablet for operation setting and configuration, user inputs, and/or display of the physiological parameters and notifications.

In one embodiment, the flow regulator comprises a stepper motor; a connection member engaged with the stepper motor and the oxygen source such that rotation of the stepper motor drives the connection member to rotate, which in turn adjusts the flow rate of oxygen from the oxygen source; and a driving circuit in communication with the controller and the stepper motor for receiving the signal from the controller and driving the stepper motor with the signal so as to adjust the flow rate of oxygen accordingly. Configuration with digital devices is utilized with this device as needed by the concentrator.

In one embodiment, the flow regulator is configured such that the flow rate is electronically driven through digital means. In other embodiments, the flow regulator comprises digital means for adjusting the flow rate of the digital concentrator electronically.

In one embodiment, the wearable device is further configured to be in wireless communication with the flow regulator.

In one embodiment, the wearable device comprises at least one pulse oximeter and/or at least one pulse co-oximeter for measuring saturation of oxygen.

In one embodiment, the wearable device further comprises at least one accelerometer for motion measurements; and/or at least one temperature sensor for temperature measurements.

In one embodiment, the controller is configured to determine whether the patient is in an active state or a rest state using output signals from the at least one accelerometer.

In one embodiment, the wearable device is configured to be worn by the patient on an ankle, a wrist, a calf, a thigh, or an upper arm.

In another aspect, the invention relates to a system for automating oxygen monitoring and dosing in real time for a patient on oxygen therapy. The system comprises a flow regulator coupled to an oxygen source for operably controlling a flow rate of oxygen from the oxygen source to the patient; and a wearable device in communication with the flow regulator and configured to be worn by the patient for measuring physiological parameters of the patient to monitor a physiological status of the patient and controlling operations of the flow regulator to regulate the flow rate of oxygen from the oxygen source for providing a desired amount of oxygen to the patient based on the physiological status of the patient.

In one embodiment, the wearable device comprises at least one pulse oximeter and/or at least one pulse co-oximeter for measuring saturation of oxygen for measuring the SpO₂ of the patient.

In one embodiment, the wearable device further comprises at least one accelerometer for motion measurements so as to determine whether the patient is in an active state or a rest state; and/or at least one temperature sensor for temperature measurements.

In one embodiment, the wearable device further comprises a controller for controlling the operations of the flow regulator based on the physiological status of the patient.

In one embodiment, when the SpO₂ is in a predetermined range, the controller generates a signal corresponding to the predetermined range and transmits the signal to the flow regulator to cause the flow regulator to adjust the flow rate of oxygen from the oxygen source corresponding to the predetermined range.

In one embodiment, the SpO₂ is measured by a bracelet pulse oximeter. To ensure accuracy of the pulse oximeter, a regular pulse and repeated SpO₂ is reassessed for 10-20 seconds, prior to the following institution of the protocol.

when the SpO₂ is in a range of 90-95%, the flow rate remains unchanged, and the SpO₂ is rechecked in a time period of 1-2 minutes;

when the SpO₂ is in a range of 85-89%, the flow rate is adjusted to increase by 1 L/min, and the SpO₂ is rechecked in a time period of 1-2 minutes;

when the SpO₂ is in a range of 81-85%, the flow rate is adjusted to increase by 2 L/min, and the SpO₂ is rechecked in a time period of 1-2 minutes;

when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1 min;

when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1-2 min; and

when the SpO₂ is greater than 96%, the flow rate is adjusted to reduce by 1 L/min, and the SpO₂ is rechecked in a time period of 2 minutes, which continues until the SpO₂ is maintained in a range of 90-95%.

In one embodiment, the controller is configured to initiate a call for help, when the patient is in a predetermined physiological status in which the SpO₂ is less than 75% for a predetermined time period, and/or shortness of breath or chest pain is present.

In one embodiment, the controller is configured to store the measured physiological parameters including the SpO₂ and the flow rate over days and weeks in local, or transmit them to a data center or a mobile device including a smartphone or tablet, which are accessible by the patient, a professional, and/or a provider.

In one embodiment, the wearable device is configured if more than three alarms occur over a course of a day, the patient is messaged to contact their provider with their oxygen desaturations.

In one embodiment, the system further comprises a customized application with a GUI installed in the wearable device for operation setting and configuration, user inputs, and/or display of the physiological parameters and notifications.

In one embodiment, the flow regulator comprises a stepper motor; a connection member engaged with the stepper motor and the oxygen source such that rotation of the stepper motor drives the connection member to rotate, which in turn adjusts the flow rate of oxygen from the oxygen source; and a driving circuit in communication with the wearable device and the stepper motor for receiving the signal from the wearable device and driving the stepper motor with the signal so as to adjust the flow rate of oxygen accordingly.

In one embodiment, the flow regulator is configured such that the flow rate of the digital concentrator is electronically driven through digital means. In other embodiments, the flow regulator comprises digital means for adjusting the flow rate of the digital concentrator electronically.

In a further aspect, the invention relates to a method for automating oxygen monitoring and dosing in real time for a patient on oxygen therapy. The method comprises continuously measuring physiological parameters of the patient, by a wearable device worn by the patient, wherein the physiological parameters comprise a SpO₂; and adjusting a flow rate of oxygen from an oxygen source, by a flow regulator coupled to the oxygen source, for providing a desired amount of oxygen to the patient based on the physiological parameters.

In one embodiment, the method further comprises generating a signal corresponding to the SPO₂, by the wearable device or a mobile device in communication with the wearable device and the flow regulator; and transmitting the signal to the flow regulator to cause the flow regulator to adjust the flow rate of oxygen from the oxygen source corresponding to the SPO₂, by the wearable device or the mobile device.

In one embodiment, the SpO₂ is measured by a bracelet pulse oximeter. To ensure accuracy of the pulse oximeter, a regular pulse and repeated SpO₂ is reassessed for 10-20 seconds, prior to the following institution of the protocol. The bracelet oximeter or physiologic measurement device determines the activity level of the patient (rest or exertion/movement) and begins the titration of oxygen as below based on the pre-set initial oxygen flow rate determined for rest and activity and prescribed by the provider. These preset starting levels for auto titration by AutO2 for rest and activity are determined based on resting measures and walks in clinic with the provider. Then, these prescribed resting and exertional starting saturations for titration are set in the algorithm. These starting titration levels for rest and exertion can be adjusted as needed as disease progresses and oxygenation worsens.

when the SpO₂ is in a range of 90-95%, the flow rate remains unchanged, and the SpO₂ is rechecked in a time period of 1-2 minutes;

when the SpO₂ is in a range of 85-89%, the flow rate is adjusted to increase by 1 L/min, and the SpO₂ is rechecked in a time period of 1-2 minutes;

when the SpO₂ is in a range of 81-85%, the flow rate is adjusted to increase by 2 L/min, and the SpO₂ is rechecked in a time period of 1-2 minutes;

when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1-2 min;

when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1-2 min; and

when the SpO₂ is greater than 96%, the flow rate is adjusted to reduce by 1 L/min, and the SpO₂ is rechecked in a time period of 2 minutes, which continues until the SpO₂ is maintained in a range of 90-95%.

In one embodiment, the method further comprises initiating a call for help, when the patient is in a predetermined physiological status in which the SpO₂ is less than 75% for a predetermined time period, and/or shortness of breath or chest pain is present.

In one embodiment, the method further comprises storing the measured physiological parameters including the SpO₂ and the flow rate over days and weeks in local, and/or transmitting them to a data center.

In one embodiment, the method further comprises notifying the patient to contact their provider with their oxygen desaturations if more than three alarms occur over a course of a day.

In one embodiment, the method further comprises generating a report that include the physiological parameters, a flow rate, alarms, time, and locations.

These and other aspects of the invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 shows schematically a system for automating oxygen monitoring and dosing in real time for a patient on oxygen therapy according to one embodiment of the invention.

FIG. 2 shows schematically a diagram of an application protocol for automating oxygen monitoring and dosing in real time for a patient on oxygen therapy according to one embodiment of the invention.

FIG. 3 shows schematically a system for automating oxygen monitoring and dosing in real time for a patient on oxygen therapy according to another embodiment of the invention.

FIG. 4 shows schematically a circuit diagram for a flow regulator according to one embodiment of the invention.

FIG. 5 shows schematically a connection member for a flow regulator according to one embodiment of the invention.

FIG. 6 shows schematically a cover box for a flow regulator according to one embodiment of the invention.

FIGS. 7A-7D show schematically pages of a customized application (AutO2 app) for automating oxygen monitoring and dosing in real time for a patient on oxygen therapy according to one embodiment of the invention.

FIG. 8 shows schematically a display of the AutO2 app according to one embodiment of the invention.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this invention, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used herein, the phrase at least one of A, B, and C should be construed to mean a logical (A or B or C), using a non-exclusive logical OR. It should be understood that one or more steps within a method may be executed in different order (or concurrently) without altering the principles of the present disclosure.

The systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

This invention discloses a device that allows for conservation of a medication, appropriate dosing of that medication and safety in medication dispensing and dose adjustment. That medication is oxygen which is a key treatment for patients with idiopathic pulmonary fibrosis (IPF). A significant advantage to IPF patients and others with hypoxic lung disease would be the peace of mind, if a device like this would provide allowing patients to have their oxygen appropriately dosed without having to think of adjusting the flow rate numerous times during the course of the date. As a patient, going about your regular daily life mentally and physically unencumbered by no longer constantly adjusting the oxygen dial should contribute significantly to quality of life improvements. With the limitations of oxygen delivery by durable medical companies, limitations in portability of oxygen and growing limitations in medical oxygen supply due to CoVID-19 related lung disease in some regions of the world, a device such as this could be invaluable for care of the acutely and chronically hypoxic patient.

This solution utilizes and marries existing technology (a wearable SpO₂ monitor, cloud based data storage) with a unique algorithm that processes the SpO₂ data and directs a novel mechanical or digital smart flow knob to adjust the oxygen flow rate accordingly. This combination of technology and algorithm coupled with data storage of SpO₂ information and needed or utilized flow rates is not present in publicly available reports or papers and certainly not utilized in the real world. Prior devices in the area have not developed appropriate algorithms that successfully supplement oxygen to keep oxygen saturations in the 90 sec for any clinically meaningful length of time. They lack consideration of concepts of physiologic delay for uptake of oxygen, circulation, and time to display of the oxygen saturation data. No remote control oxygen delivery device is presently in the marketplace for patients.

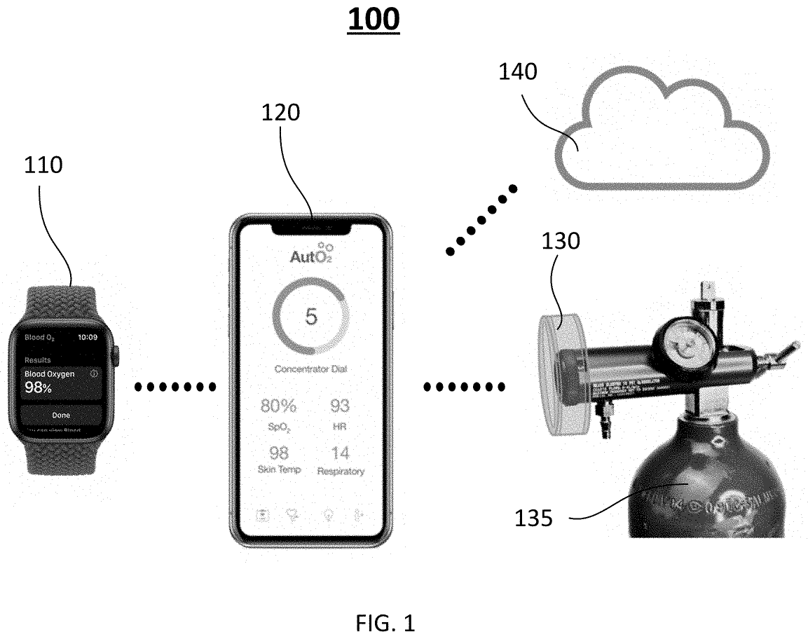

Referring to FIG. 1, a system 100 for automating oxygen monitoring and dosing in real time for a patient on oxygen therapy is shown according to one embodiment of the invention. When a patient is on oxygen therapy, the patient is administrated with an amount/dose of oxygen usually by a tube connecting between the patient and an oxygen source such as an oxygen tank. The amount/dose of oxygen should be adjustable based on patient's physiological need or status in real time. The physiological need or status can be evaluated or determined by patient's physiological parameters in real time, such as, but are not limited to, a SpO₂, a heart rate, a respiratory rate, a temperature, activity level, and/or a blood pressure.

In the exemplary embodiment shown in FIG. 1, the system 100 includes a flow regulator 130 coupled to an oxygen source 135 for operably controlling a flow rate of oxygen from the oxygen source 135 to the patient; a wearable device 110 configured to be worn by the patient for measuring physiological parameters of the patient to monitor a physiological status of the patient; and a controller 120 in wireless communication with the wearable device 110 and the flow regulator 130 for receiving the measured physiological parameters from the wearable device 110, and controlling operations of the flow regulator 130 to regulate the flow rate of oxygen from the oxygen source 135 for providing an desired amount of oxygen to the patient based on the physiological status of the patient. The wireless communication is performed via the internet, WiFi, API, and/or Bluetooth protocols.

The flow regulator 130 is a mechanical or digital smart flow knob that is operably engaged with a standard oxygen concentrator/tank 135 and is capable of automatically adjusting the oxygen flow rate from the oxygen tank 135 to the patient according to the patient's physiological status that is determined by the measured physiological parameters. In some embodiments, the flow regulator 130 comprises a stepper motor; a connection member engaged with the stepper motor and the oxygen source 135 such that rotation of the stepper motor drives the connection member to rotate, which in turn adjusts the flow rate of oxygen from the oxygen source 135; and a driving circuit in communication with the controller 120 and the stepper motor for receiving the signal from the controller 120 and driving the stepper motor with the signal so as to adjust the flow rate of oxygen accordingly. In one embodiment, the driving circuit includes an API/Bluetooth module/circuit/driver for the wireless communication with the controller 120, and/or the wearable device 110.

In other embodiments, some portable oxygen concentrators and home oxygen concentrators are digital and electronically driven instead of driven or adjusted by a rotary knob. For the digital concentrators, accessibility for adjustment is included in this concept. For example, the flow regulator is configured such that the flow rate of the digital concentrators is electronically driven through digital means. In other embodiments, the flow regulator comprises digital means for adjusting the flow rate of the digital concentrators electronically.

The wearable device 110 is essentially a sensor device that includes one or more pulse oximeters, and/or one or more pulse co-oximeter for measuring the SpO₂ of the patient.

In some embodiments, the wearable device 110 may also include at least one accelerometer for motion measurements for evaluating a rest or active (e.g., walking) state of the patient; and/or at least one temperature sensor for temperature measurements.

In some embodiments, the wearable device 110 is further configured to be in wireless communication with the flow regulator 130.

In some embodiments, the wearable device 110 is configured to be worn by the patient on an ankle, a wrist, a calf, a thigh, an upper arm, or other applicable body part for oxygen saturation detection.

In one embodiment, the wearable device 110 may also include an API/Bluetooth module/circuit/driver for the wireless communication with the controller 120, and/or the flow regulator 130.

The controller 120 is wirelessly coupled with the wearable device 110 and the flow regulator 130. It is adapted for receiving the measured physiological parameters from the wearable device 110 and controlling operations of the flow regulator 130 to regulate the flow rate of oxygen from the oxygen source 135 for providing a desired amount of oxygen to the patient based on the physiological status of the patient. Specifically, when the physiological parameter, e.g., the SpO₂, is received from the wearable sensor 110, the controller 120 first determines if the SpO₂ is in a predetermined range and what the activity level is (rest or exertion/movement), and then generates a signal based on the predetermined range and transmits the signal to the flow regulator 130 to drive the flow regulator 130 to adjust the flow rate of oxygen from the oxygen source corresponding to the predetermined range. The bracelet oximeter or physiologic measurement device determines the activity level of the patient (rest or exertion/movement) and begins the titration of oxygen as below based on the pre-set initial oxygen flow rate determined for rest and activity and prescribed by the provider. These preset starting levels for auto titration by AutO2 (a customized application) for rest and activity are determined based on resting measures and walks in clinic with the provider. Then, these prescribed resting and exertional starting saturations for titration are set in the algorithm. These starting titration levels for rest and exertion can be adjusted as needed as disease progresses and oxygenation worsens.

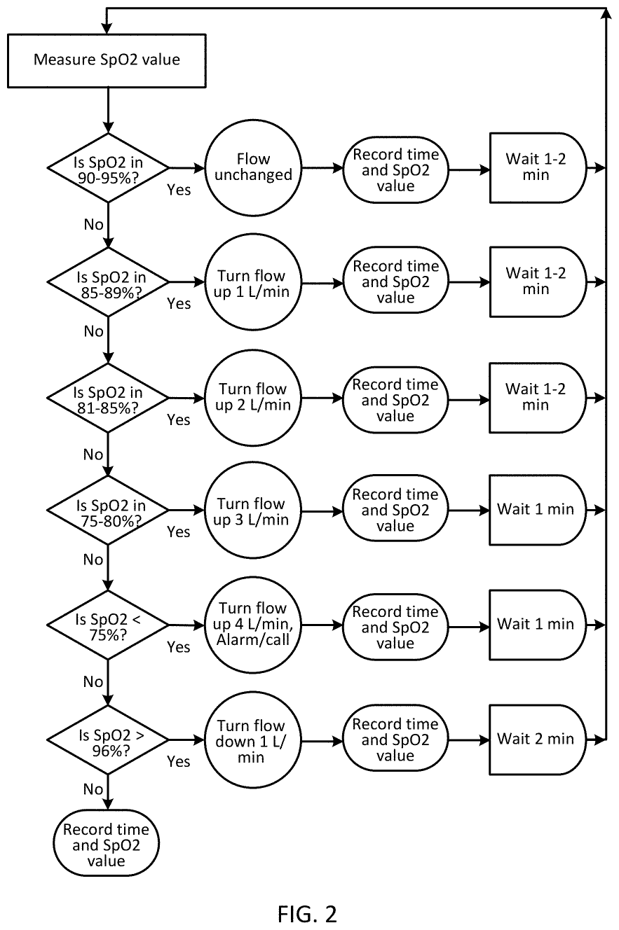

For example, as shown in FIG. 2, when the SpO₂ is in a range of 90-95%, the flow rate remains unchanged, and the SpO₂ is rechecked in a time period of 1-2 minutes;

when the SpO₂ is in a range of 85-89%, the flow rate is adjusted to increase by 1 L/min, and the SpO₂ is rechecked in a time period of 1-2 minutes;

when the SpO₂ is in a range of 81-85%, the flow rate is adjusted to increase by 2 L/min, and the SpO₂ is rechecked in a time period of 1-2 minutes;

when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1-2 min;

when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1-2 min; and

when the SpO₂ is greater than 96%, the flow rate is adjusted to reduce by 1 L/min, and the SpO₂ is rechecked in a time period of 2 minutes, which continues until the SpO₂ is maintained in a range of 90-95%.

To ensure accuracy of the pulse oximeter, a regular pulse and repeated SpO₂ is reassessed about 10 seconds later, prior to the above protocol.

In some embodiments, the controller 120 is configured to initiate a call for help, when the patient is in a predetermined physiological status in which the SpO₂ is less than 75% for a predetermined time period, and/or shortness of breath or chest pain is present.

In some embodiments, the controller is configured to average the measured physiological parameters of the patient over a time period of 10-20 sec.

In some embodiments, the controller 120 is configured if more than three alarms occur over a course of a day, the patient is messaged to contact their provider with their oxygen desaturations.

In some embodiments, the controller 120 is configured to store the measured physiological parameters including the SpO₂ and the flow rate over days and weeks in local, or transmit them to a data center, which are accessible by the patient, a professional, and/or a provider.

In one embodiment, the controller 120 is configured to determine whether the patient is in an active state or a rest state using output signals from the at least one accelerometer.

In some embodiments, the controller 120 is a smartphone, a tablet, or any type of smart mobile devices. A customized application with a graphic user interface (GUI) is installed in the smartphone or tablet for operation setting and configuration, user inputs, display of the physiological parameters and notifications, and so on.

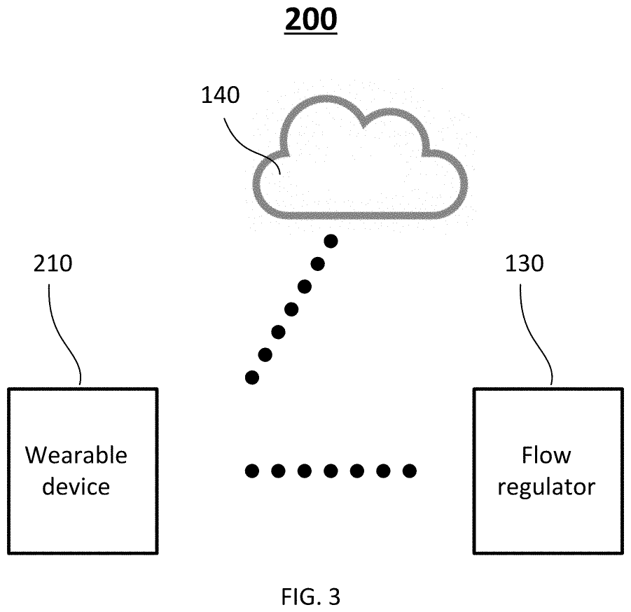

Referring to FIG. 3, a system 200 for automating oxygen monitoring and dosing in real time for a patient on oxygen therapy is shown according to another embodiment of the invention. The system 200 includes a flow regulator 130 coupled to an oxygen source for operably controlling a flow rate of oxygen from the oxygen source to the patient; and a wearable device 210 in communication with the flow regulator 130 and configured to be worn by the patient for measuring physiological parameters of the patient to monitor a physiological status of the patient and controlling operations of the flow regulator 130 to regulate the flow rate of oxygen from the oxygen source for providing an desired amount of oxygen to the patient based on the physiological status of the patient.

In some embodiments, the wearable device 210 comprises at least one pulse oximeter and/or at least one pulse co-oximeter for measuring saturation of oxygen for measuring the SpO₂ of the patient.

In some embodiments, the wearable device 210 further comprises at least one accelerometer for motion measurements so as to determine whether the patient is in an active state or a rest state; and/or at least one temperature sensor for temperature measurements.

In some embodiments, the wearable device 210 further comprises a controller for controlling the operations of the flow regulator 130 based on the physiological status of the patient.

In some embodiments, the wearable device 210 is essentially an integration of a sensor device 110 and a controller 120 of the system 100, and the system 200 has the same functions as that of the system 100, as described above.

According to embodiments of the invention, the system for automating oxygen monitoring and dosing in real time for a patient on oxygen therapy is an automated system and capable of connecting to industry standard oxygen concentrators/tanks, sensing patient's oxygen saturation in real time, and adjusting the flow rate according to the patient's needs to maintain appropriate oxygenation, reducing oxygen waste and costs, and eliminating the hassle of oxygen titration. The system creates a closed loop system that makes adjustments of oxygen based changes in the skin sensors, which yields profoundly better results in patient outcome.

In one aspect, the invention also relates to a method for automating oxygen monitoring and dosing in real time for a patient on oxygen therapy. The method comprises continuously measuring the patient's physiological parameters such as SpO₂ by a wearable device worn by the patient and adjusting a flow rate of oxygen from an oxygen source, by a flow regulator coupled to the oxygen source, for providing an desired amount of oxygen to the patient based on the SpO₂ and activity level.

In some embodiments, the method further comprises generating a signal corresponding to the SpO₂ and activity level, by the wearable device or a mobile device in communication with the wearable device and the flow regulator; and transmitting the signal to the flow regulator to cause the flow regulator to adjust the flow rate of oxygen from the oxygen source corresponding to the SpO₂, by the wearable device or the mobile device. For example, when the SpO₂ is in a range of 90-95%, the flow rate remains unchanged, and the SpO₂ is rechecked in a time period of 1-2 minutes; when the SpO₂ is in a range of 85-89%, the flow rate is adjusted to increase by 1 L/min, and the SpO₂ is rechecked in a time period of 1-2 minutes; when the SpO₂ is in a range of 81-85%, the flow rate is adjusted to increase by 2 L/min, and the SpO₂ is rechecked in a time period of 1-2 minutes; when the SpO₂ is in a range of 75-80%, an alarm is triggered, the patient is messaged to sit down/rest, and the flow rate is adjusted to increase by 3 L/min or to a maximum flow rate if 3 L/min is lower than the maximum flow rate, and the SpO₂ is rechecked in a time period of 1 min; when the SpO₂ is less than 75%, an alarm is triggered, the patient is messaged to sit down/rest, and the flow rate is adjusted to increase by 4 L/min or to the maximum flow rate if 4 L/min is lower than the maximum flow rate, and the SpO₂ is rechecked in a time period of 1 min; and when the SpO₂ is greater than 96%, the flow rate is adjusted to reduce by 1 L/min, and the SpO₂ is rechecked in a time period of 2 minutes, which continues until the SpO₂ is maintained in a range of 90-95%. The starting point of flow rate for titration is based on activity level as determined by the wearable device.

In some embodiments, the method further comprises initiating a call for help (e.g., 911 call), when the patient is in a predetermined physiological status in which the SpO₂ is less than 75% for a predetermined time period, and/or shortness of breath or chest pain is present.

In some embodiments, the method further comprises storing the measured physiological parameters including the SpO₂ and the flow rate over days and weeks in local, and/or transmitting them to a data center.

In some embodiments, the method further comprises noticing the patient to contact their provider with their oxygen desaturations if more than three alarms occur over a course of a day.

In some embodiments, the method further comprises generating a report that include the physiological parameters, a flow rate, alarms, time, and locations.

It should be noted that all or a part of the steps according to the embodiments of the invention is implemented by hardware or a program instructing relevant hardware. Yet another aspect of the invention provides a non-transitory tangible computer-readable medium storing instructions which, when executed by one or more processors, perform the above method for automating oxygen monitoring and dosing in real time for a patient on oxygen therapy. The computer executable instructions or program codes enable a computer or a similar computing system to complete various operations in the above disclosed method for privilege management. The storage medium/memory may include, but is not limited to, high-speed random access medium/memory such as DRAM, SRAM, DDR RAM or other random access solid state memory devices, and non-volatile memory such as one or more magnetic disk storage devices, optical disk storage devices, flash memory devices, or other non-volatile solid state storage devices.

Without intent to limit the scope of the invention, examples and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Flow Regulator (Smart Knob)

The flow regulator in one embodiment of the invention is a mechanical or digital smart flow knob that is operably engaged with a standard oxygen concentrator/tank for automatically adjusting the oxygen flow rate from the oxygen tank to the patient according to the patient's physiological status that is determined by the measured physiological parameters. Since the flow rate on most oxygen concentrators is controlled by the twisting of a knob, a general design that can change its size can be created to attach to the knob and then the device can be manually calibrated according to the sensitivity of the oxygen concentrator.

The flow regulator has a stepper motor; a connection member engaged with the stepper motor and the oxygen source such that rotation of the stepper motor drives the connection member to rotate, which in turn adjusts the flow rate of oxygen from the oxygen source; and a driving circuit in communication with the controller and the stepper motor for receiving the signal from the controller and driving the stepper motor with the signal so as to adjust the flow rate of oxygen accordingly.

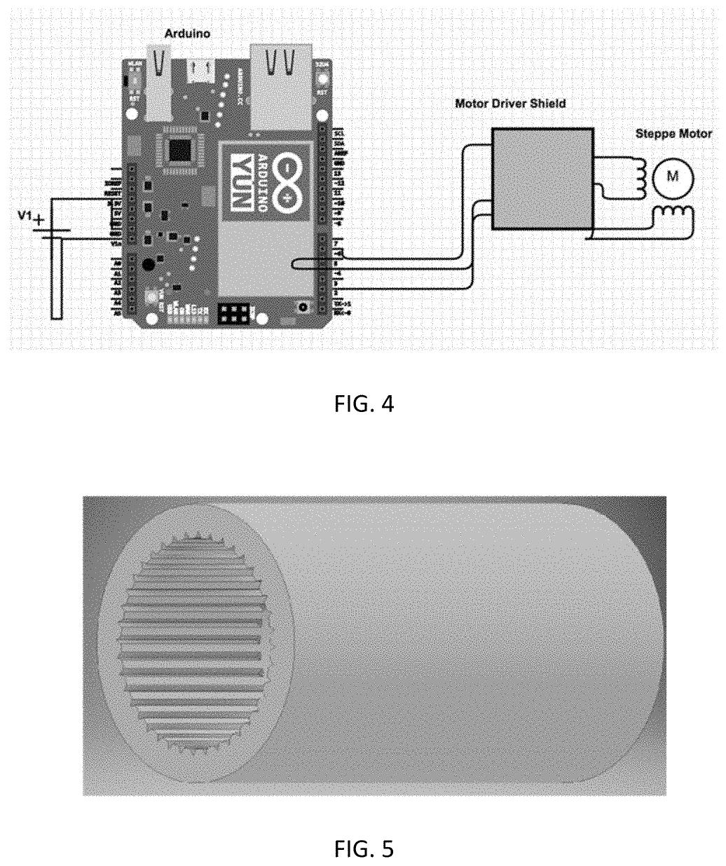

As shown in FIG. 4, a schematic circuit diagram used for the mechanistic driver within the flow regulator is shown according to one embodiment of the invention. The circuit includes an Arduino board, a breadboard, assorted wires, a Bluetooth driver, a stepper motor driver, and a stepper motor. The stepper motor is engaged with a connection member that is in turn connected to an oxygen tank/concentrator. The connection member is configured to allow the flow regulator to transfer motor's rotation to a knob of the oxygen tank.

As shown in FIG. 5, in one embodiment the connection member is a nylon cylinder with ridges on the interior. The ridges on the cylinder match those on the oxygen tank allowing it to fit directly on to the oxygen tank knob. The other side of the cylinder connects to the stepper motor thereby transferring the inertia from the stepper motor to the oxygen tank knob.

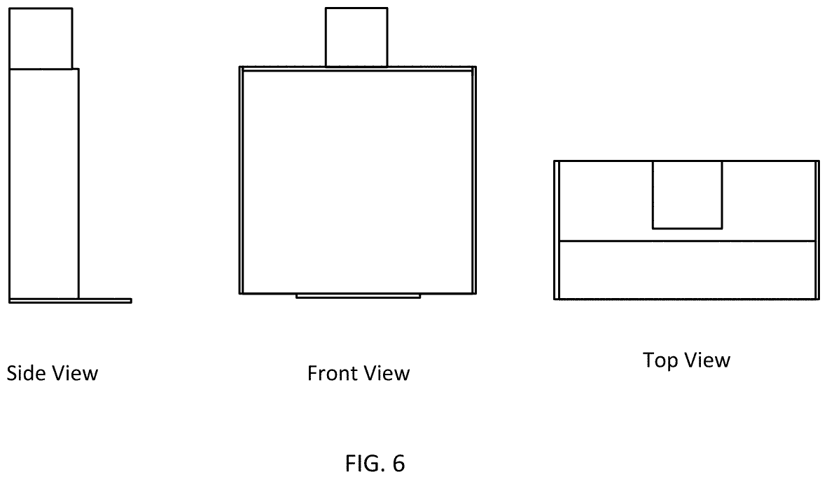

The mechanistic driver is contained in an acrylic box atop an acrylic shield that can be placed next to the oxygen tank. The acrylic structure is shown FIG. 6. The top section of the side and front views shows the box where the mechanistic driver and all of its components are placed. The rest of the structure is placed near the oxygen tank and provide support for the mechanistic driver.

The flow regulator (device) is able to attach to and alter flow on existing oxygen concentrators, meaning that no new concentrators need to be made for this device. The user also has the ability to remove the device if errors occur and the system is able to be calibrated to the sensitivity of the oxygen concentrator it is being used on.

In other embodiments, some portable oxygen concentrators and home oxygen concentrators are digital and electronically driven instead of driven or adjusted by a rotary knob. For digital concentrators, accessibility for adjustment is included in this concept. For example, in some embodiments, the flow regulator is configured such that the flow rate of the digital concentrators is electronically driven through digital means. In other embodiments, the flow regulator comprises digital means for adjusting the flow rate of the digital concentrators electronically.

### Example 2

### Algorithm and Application Protocol

According to embodiments of the invention, the algorithm and application protocol provides improved oximeters by using wrist pulse oximetry, and multi wavelength pulse co-oximetry, instead of finger and earlobe pulse oximetry, and averaging the reading from the pulse oximeter over about 10-20 sec.

The algorithm and application protocol also provides the oxygen flow rate adjusted according to the patient's SpO₂ per protocol with feedback by, for example, a proportional-integral-derivative (PID) system with mass flow regulator allowing for up to 20 L/min. Reading from the pulse oximeter is averaged over about 10-20 sec.

The algorithm and application protocol allows time for accurate reading/discriminating error and artifact. Each circulation of a red blood cell (RBC) takes about 60 seconds. SpO₂ readings sent to adjust the flow rate too close together do not allow time for circulation or accurate assessment with adjustment of the flow rate. Most devices capture the SpO₂ signal every second with rapid adjustment of the flow rate. This can result in over and under titration of oxygen in patients. Most automated systems in the public space show up to 22% desaturation during the measured time of use.

The algorithm and application protocol also allows to set the parameter of starting flow rates for O₂ at rest and with exertion, so as to minimize the time needed to get to the final best flow rate with fewer O₂ flow rate titrations to keep saturations at 90% or better with rest or exertion. Rest to exertion and exertion to rest trigger changes in the actigraphy that is transmitted to the algorithm.

The algorithm and application protocol has the ability of the pulse oximetry to send a signal directly to a phone and the flow regulator. If the phone battery dies, the signal can divert to the flow regulator so that oxygen titration is not interrupted.

The algorithm and application protocol can also alert patients if signals are lost from wearables or phone, low sustained saturations occur for 60 seconds or longer, and/or high sustained pulse rate (>140) and low pulse rate (<40).

The algorithm and application protocol has the ability for providers to set alert parameters and resting/exertional flow rates, message the patient/the primary care physician (PCP)/pulmonologist/pulmonologist's triage nurse if the patient's flow rates and oxygen needs are increasing over the past week or set time period. These parameters with messaging endpoint can be set by providers. Data acquisition of needed flow rates and saturation occur over the prior week or month via online database—similar to CPAP downloads of compliance and AHI data.

The algorithm and application protocol also provides security with password protected entry of prescribed parameters, and password protection for data acquisition by providers.

Most devices do not take error or artifact of the oximeter into account. Multi wavelength pulse co-oximetry is utilized to improve sensing. The reading from pulse oximeter should be averaged over 10-20 sec before use in algorithm to avoid reading errors with artifact and drop out of pulse and SpO₂. The wearable sends SpO₂ and actigraphy data to the application on the smartphone. The oxygen flow rate is adjusted according to the SpO₂ per protocol within the app with feedback. Most devices capture the SpO₂ every second which is too quickly for human physiology to respond. Most automated systems show % desaturation of up to 22% of the testing time. This device takes the risk of artifact and human physiologic delay into account in the algorithm.

A PID system with mass flow regulator allows for adjustment of flow rate up to 20 L/min.

For the flow regulator (Smartknob), parameters of the starting oxygen flow rate are set at rest and with exertion. For example: 2 L/min at rest and 6 L/min with exertion. Set resting and exertional flow rates as a starting point for oxygen titration allow for large jumps to occur with changes in activity level up or down with smaller titrations from the set resting and exertional flow rates. This will minimize the time need to get to the final best flow rate w fewer titration keep saturations in 90s with exertion or rest. Rest to exertion and exertion to rest triggers recording of movement or rest with actigraphy incorporated in the wearable. App sends signal to Smartknob on oxygen regulator to adjust flow rate.

In some embodiments, the provider sets the “resting” and “exertional” or “activity” flow rate. The App algorithm begins titration from set flow rates. Actigraphy identifies if patient is at “rest” or “exertion”. If at rest or sitting, titration begins at set “resting” flow rate. If moving, active or exerting, titration begins at set “activity” flow rate. One exemplary example of the algorithm is as follows:
If the SpO₂ is in the range of 90-95%, then the flow rate remains unchanged.
If the SpO₂ is the range of 87-89%, then the flow rate is increased by 1 L/min.
If the SpO₂ is the range of 83-86%, then the flow rate is increased by 2 L/min.
If the SpO₂ is the range of 79-82%, then the flow rate increases by 3 L/min and trigger alarm noting low oxygen saturation.
If the SpO₂ is <79%, the flow rate increases to max flow with audible alarm advising patient to rest, call for assistance or 911.
If the SpO₂>95%, then the flow rate decreases by 1 L/min.

The SpO₂ is measured with a moving window of 10-20 seconds that identifies a consistent pulse (to ensure good signal) and averages continuously over the prior 10-20 seconds. The SpO₂ is continuously detected with the prior 10-20 seconds averaged by the AutO2 app.

### Example 3

### AutO2 App with User Interface

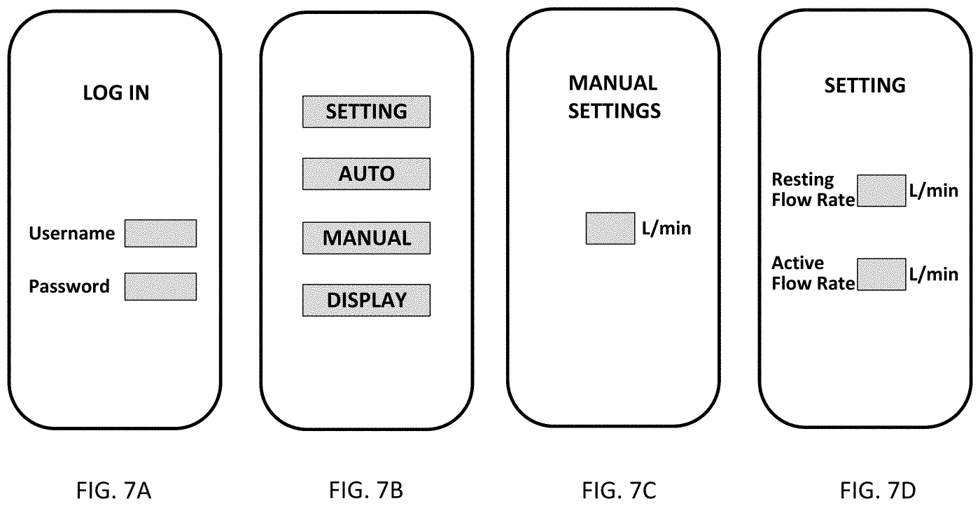

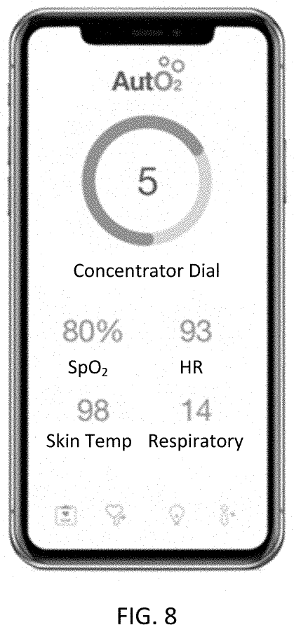

FIGS. 7A-7D and 8 show one embodiment of a customized application (AutO2 app) with a GUI installed in the smartphone for operation setting and configuration, user inputs, display of the physiological parameters and notifications, and so on.

FIGS. 7A-7D show a few of pages of the AutO2 app for operation setting and configuration and user inputs, as an example. It should be appreciated that the AutO2 app can also be presented in other forms. The first page of the AutO2 app show in FIG. 7A is for logging in, where username and password are required to initiate the AutO2 app. The new few pages shown in FIGS. 7B-7D are for user inputs of the blood O₂ saturation level in addition to manually inputting the oxygen liter flow rate the patient requires.

For example, the second page shown in FIG. 7B lists a number of confirmations, such as “SETTING”, “AUTO”, “MANUAL” and “DISPLAY”. When “AUTO” is selected, the application will automatically set resting and exertional flow rates based on the actigraphy reading of rest vs exertion. When “MANUAL” is selected, the application will allow the patient to set the flow rate at this moment, as shown in FIG. 7C. This is a safety hatch so that if the patient desaturates and needs to adjust to a maximal flow rate of oxygen, they can. When “SETTING” is selected at the fourth page shown in FIG. 7D, the provider can set “Resting Flow Rate”—flow rate needed to keep saturations in 90 sec at rest, and “Active Flow Rate”—flow rate needed to keep saturations in 90 sec during activity, like walking. In some embodiment, scrolling wheel with numbers up to 20 L/min is adapted for setting resting and active flow rates. The app should sense a transition to activity by the actigraphy monitor on the wearable device and increase the flow rate to the active level to allow a quicker adjustment of the flow rate to keep saturations in the 90 sec with activity. When “DISPLAY” is selected, the application will display SpO₂, HR, Skin Temperature, Respiratory rate and Flow rate. One exemplary display is shown in FIG. 8.

In conclusion, the system for pulse oximeter saturation monitors and API/Bluetooth devices to adjust a knob (like the knob on an oxygen regulator) are disclosed. The unique feature of the system and method is the combination of monitoring coupled with a unique protocol adapted into an app to send the correct flow rate signal via Bluetooth to the oxygen regulator once the oxygen saturation has been interpreted by the pulse oximeter. No one has linked these elements with a unique smart oxygen auto adjusting protocol. This unique know how is the link between measurement of oxygen saturation and deployment of the correct dosing of oxygen. The system provides automated oxygen monitoring and dosing in real time allowing patients wearing oxygen to no longer need to constantly think about adjusting their oxygen flow rates as their activity level changes.

Other applications of the device include deployment in the hospital environment. This is perhaps an even more important application for patient safety. Presently, patients on oxygen in hospitals are adjusted by the nurse who may notice alarms for low oxygen levels or tachycardia and respond by leaving one patient's room and moving to the one with alarms sounding. Due to location and the duties the nurse is performing with another patient, a time delay in adjustment of oxygen occurs. A real time device that sounds the alarm while adjusting the oxygen per software protocol to the flow rate needed by the patient to maintain normoxia would correct hypoxia sooner while avoiding lost time and the numerous complications of hypoxia most notably arrythmia with cardiopulmonary arrest in the acutely ill patient.

As we transition to telehealth visits in this post CoVID-19 era, data on oxygenation and respiratory status in the home is lacking. Having objective data from the home via this device could provide an additional element of objective data to these visits and possibly provide more helpful information than our six minute walk tests or other clinic walk tests can provide.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

What is claimed is:

1. A system for automating oxygen monitoring and dosing in real time for a patient on oxygen therapy, comprising:
a flow regulator coupled to an oxygen source for operably controlling a flow rate of oxygen from the oxygen source to the patient;
a wearable device configured to be worn by the patient for measuring physiological parameters of the patient to monitor a physiological status of the patient; and
a controller in wireless communication with the wearable device and the flow regulator for receiving the measured physiological parameters from the wearable device, and controlling operations of the flow regulator to regulate the flow rate of oxygen from the oxygen source for providing a desired amount of oxygen to the patient based on the physiological status of the patient.

2. The system of claim 1, wherein the physiological parameters comprises at least one of an oxygen saturation (SpO₂), a heart rate, a respiratory rate, a temperature, activity level and a blood pressure.

3. The system of claim 2, wherein when the SpO₂ is in a predetermined range, the controller generates a signal corresponding to the predetermined range and transmits the signal to the flow regulator to cause the flow regulator to adjust the flow rate of oxygen from the oxygen source corresponding to the predetermined range based on the measured SpO₂ and app algorithm.

4. The system of claim 3, wherein
when the SpO₂ is in a range of 90-95%, the flow rate remains unchanged, and the SpO₂ is rechecked in a time period of 1-2 minutes;
when the SpO₂ is in a range of 85-89%, the flow rate is adjusted to increase by 1 L/min, and the SpO₂ is rechecked in a time period of 1-2 minutes;
when the SpO₂ is in a range of 81-85%, the flow rate is adjusted to increase by 2 L/min, and the SpO₂ is rechecked in a time period of 1-2 minutes;
when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1-2 min;
when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1-2 min; and
when the SpO₂ is greater than 96%, the flow rate is adjusted to reduce by 1 L/min, and the SpO₂ is rechecked in a time period of 2 minutes, which continues until the SpO₂ is maintained in a range of 90-95%.

5. The system of claim 4, wherein the controller is configured to initiate a call for help, when the patient is in a predetermined physiological status in which the SpO₂ is less than 75% for a predetermined time period, and/or shortness of breath or chest pain is present.

6. The system of claim 4, wherein the controller is configured to store the measured physiological parameters including the SpO₂ and the flow rate over days and weeks in local, or transmit them to a data center, which are accessible by the patient, a professional, and/or a provider.

7. The system of claim 4, being configured if more than three alarms occur over a course of a day, the patient is messaged to contact their provider with their oxygen desaturations.

8. The system of claim 1, wherein the controller is configured to average the measured physiological parameters of the patient over a time period of 10-20 sec.

9. The system of claim 1, wherein the controller is a smartphone or tablet.

10. The system of claim 9, further comprising a customized application with a graphic user interface (GUI) installed in the smartphone or tablet for operation setting and configuration, user inputs, and/or display of the physiological parameters and notifications.

11. The system of claim 1, wherein the flow regulator comprises:
a stepper motor;
a connection member engaged with the stepper motor and the oxygen source such that rotation of the stepper motor drives the connection member to rotate, which in turn adjusts the flow rate of oxygen from the oxygen source; and
a driving circuit in communication with the controller and the stepper motor for receiving the signal from the controller and driving the stepper motor with the signal so as to adjust the flow rate of oxygen accordingly.

12. The system of claim 1, wherein the flow regulator is configured such that the flow rate is electronically driven through digital means.

13. The system of claim 1, wherein the wearable device is further configured to be in wireless communication with the flow regulator.

14. The system of claim 1, wherein the wearable device comprises at least one pulse oximeter and/or at least one pulse co-oximeter for measuring saturation of oxygen.

15. The system of claim 14, wherein the wearable device further comprises at least one accelerometer for motion measurements; and/or at least one temperature sensor for temperature measurements.

16. The system of claim 15, wherein the controller is configured to determine whether the patient is in an active state or a rest state using output signals from the at least one accelerometer.

17. The system of claim 1, wherein the wearable device is configured to be worn by the patient on an ankle, a wrist, a calf, a thigh, an upper arm or other body part capable of SpO₂ detection.

18. A system for automating oxygen monitoring and dosing in real time for a patient on oxygen therapy, comprising:
a flow regulator coupled to an oxygen source for operably controlling a flow rate of oxygen from the oxygen source to the patient; and
a wearable device in communication with the flow regulator and configured to be worn by the patient for measuring physiological parameters of the patient to monitor a physiological status of the patient and controlling operations of the flow regulator to regulate the flow rate of oxygen from the oxygen source for providing an desired amount of oxygen to the patient based on the physiological status of the patient.

19. The system of claim 18, wherein the wearable device comprises at least one pulse oximeter and/or at least one pulse co-oximeter for measuring saturation of oxygen for measuring an oxygen saturation (SpO₂) of the patient.

20. The system of claim 19, wherein the wearable device further comprises at least one accelerometer for motion measurements so as to determine whether the patient is in an active state or a rest state; and/or at least one temperature sensor for temperature measurements.

21. The system of claim 20, wherein the wearable device further comprises a controller for controlling the operations of the flow regulator based on the physiological status of the patient.

22. The system of claim 21, wherein when the SpO₂ is in a predetermined range, the controller generates a signal corresponding to the predetermined range and transmits the signal to the flow regulator to cause the flow regulator to adjust the flow rate of oxygen from the oxygen source corresponding to the predetermined range.

23. The system of claim 22 wherein
when the SpO₂ is in a range of 90-95%, the flow rate remains unchanged, and the SpO₂ is rechecked in a time period of 1-2 minutes;
when the SpO₂ is in a range of 85-89%, the flow rate is adjusted to increase by 1 L/min, and the SpO₂ is rechecked in a time period of 1-2 minutes;
when the SpO₂ is in a range of 81-85%, the flow rate is adjusted to increase by 2 L/min, and the SpO₂ is rechecked in a time period of 1-2 minutes;
when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1-2 min;
when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1-2 min; and
when the SpO₂ is greater than 96%, the flow rate is adjusted to reduce by 1 L/min, and the SpO₂ is rechecked in a time period of 2 minutes, which continues until the SpO₂ is maintained in a range of 90-95%.

24. The system of claim 23, wherein the controller is configured to initiate a call for help, when the patient is in a predetermined physiological status in which the SpO₂ is less than 75% for a predetermined time period, and/or shortness of breath or chest pain is present.

25. The system of claim 23, wherein the controller is configured to store the measured physiological parameters including the SpO₂ and the flow rate over days and weeks in local, or transmit them to a data center or a mobile device including a smartphone or tablet, which are accessible by the patient, a professional, and/or a provider.

26. The system of claim 23, wherein the wearable device is configured if more than three alarms occur over a course of a day, the patient is messaged to contact their provider with their oxygen desaturations.

27. The system of claim 18, further comprising a customized application with a graphic user interface (GUI) installed in the wearable device for operation setting and configuration, user inputs, and/or display of the physiological parameters and notifications.

28. The system of claim 18, wherein the flow regulator comprises:
a stepper motor;
a connection member engaged with the stepper motor and the oxygen source such that rotation of the stepper motor drives the connection member to rotate, which in turn adjusts the flow rate of oxygen from the oxygen source; and
a driving circuit in communication with the wearable device and the stepper motor for receiving the signal from the wearable device and driving the stepper motor with the signal so as to adjust the flow rate of oxygen accordingly.

29. The system of claim 18, wherein the flow regulator is configured such that the flow rate is electronically driven through digital means.

30. A method for automating oxygen monitoring and dosing in real time for a patient on oxygen therapy, comprising:
continuously measuring physiological parameters of the patient, by a wearable device worn by the patient, wherein the physiological parameters comprise an oxygen saturation (SpO₂); and
adjusting a flow rate of oxygen from an oxygen source, by a flow regulator coupled to the oxygen source, for providing an desired amount of oxygen to the patient based on the physiological parameters.

31. The method of claim 30, further comprising:
generating a signal corresponding to the SPO₂, by the wearable device or an mobile device in communication with the wearable device and the flow regulator; and
transmitting the signal to the flow regulator to cause the flow regulator to adjust the flow rate of oxygen from the oxygen source corresponding to the SPO₂, by the wearable device or the mobile device.

32. The method of claim 31, wherein
when the SpO₂ is in a range of 90-95%, the flow rate remains unchanged, and the SpO₂ is rechecked in a time period of 1-2 minutes;
when the SpO₂ is in a range of 85-89%, the flow rate is adjusted to increase by 1 L/min, and the SpO₂ is rechecked in a time period of 1-2 minutes;
when the SpO₂ is in a range of 81-85%, the flow rate is adjusted to increase by 2 L/min, and the SpO₂ is rechecked in a time period of 1-2 minutes;
when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1-2 min;
when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1 min; and
when the SpO₂ is greater than 96%, the flow rate is adjusted to reduce by 1 L/min, and the SpO₂ is rechecked in a time period of 2 minutes, which continues until the SpO₂ is maintained in a range of 90-95%.

33. The method of claim 32, further comprising initiating a call for help, when the patient is in a predetermined physiological status in which the SpO₂ is less than 75% for a predetermined time period, and/or shortness of breath or chest pain is present.

34. The method of claim 32, further comprising storing the measured physiological parameters including the SpO₂ and the flow rate over days and weeks in local, and/or transmitting them to a data center.

35. The method of claim 32, further comprising noticing the patient to contact their provider with their oxygen desaturations if more than three alarms occur over a course of a day.

36. The method of claim 30, further comprising generating a report that include the physiological parameters, a flow rate, alarms, time, and locations.
